(19) United States
(12) Patent Application Publication
(10) Pub. No.: US 2026/0076883 A1
(43) Pub. Date: Mar. 19, 2026

(54) DRY SHAMPOO AND RELATED METHODS

(71) Applicant: PLZ Corp., Downers Grove, IL (US)

(72) Inventors: Ian Joshua FISHMAN, Riverside, CA (US); Jorge Alfredo POLANCO, Riverside, CA (US); Alfonso Cecilio MORENO, Riverside, CA (US)

(21) Appl. No.: 19/326,163

(22) Filed: Sep. 11, 2025

Related U.S. Application Data
(60) Provisional application No. 63/694,379, filed on Sep. 13, 2024.

(51) Int. Cl. A61K 8/33; A61K 8/25; A61K 8/26; A61K 8/67; A61K 8/73; A61Q 5/02

(52) U.S. Cl. CPC A61K 8/33; A61K 8/25; A61K 8/26; A61K 8/673; A61K 8/732; A61Q 5/02; A61K 2800/30

(57) ABSTRACT

This disclosure relates to dry shampoo compositions. In particular, the present disclosure pertains to dry shampoo compositions that are environmentally friendly yet effective. The dry shampoo compositions provided herein may function, in part, by absorbing excess oil and sebum from the scalp and hair, thereby reducing the appearance of greasiness and extending the time between conventional washes.

### CROSS-REFERENCE TO RELATED APPLICATIONS

The present application claims priority to U.S. Application No. 63/694,379 filed Sep. 13, 2024, the contents of which are incorporated herein.

### BACKGROUND

Dry shampoos have gained widespread popularity as a convenient alternative to traditional shampoos, allowing users to refresh their hair without needing water. Originally developed as a time-saving alternative to traditional shampoo, dry shampoo absorbs excess oil and adds volume to hair without water. While dry shampoo formulations have evolved significantly with recent advancements focusing on enhancing formulation effectiveness, there remains a need, driven by both consumer demand and regulatory changes, to provide products that also meet environmental standards.

Some dry shampoo formulations feature natural and organic ingredients, which cater to the rising consumer preference for clean beauty products. For example, some dry shampoo formulations incorporate plant-based ingredients and avoid harsh chemicals like sulfates and parabens. There is also an ongoing shift towards water-less formulations, which are more sustainable since they reduce water use during production and require less packaging. Additionally, the trend of offering customizable formulations is gaining traction, allowing users to select dry shampoos tailored to their specific hair type and needs.

In addition to ingredient reformulation, there is a broader trend towards sustainability across the personal care industry. Consumers are demanding products that are not only effective but also environmentally responsible. As a result, dry shampoos are often free from sulfates, parabens, and other harmful chemicals, with an increased focus on using natural and biodegradable ingredients. These changes are aligned with the clean beauty movement, which prioritizes transparency and safety in product formulations.

Dry shampoos that include high concentrations of ingredients like alcohols (e.g., ethanol) and hydrofluorocarbons (e.g., HFC-152a) may be efficient, but contribute to a higher environmental impact. With recent changes, there's a shift toward using more eco-friendly alternatives that still deliver the desired results.

One critical aspect influencing modern formulations is the Maximum Incremental Reactivity (MIR) metric. MIR quantifies the potential of volatile organic compounds (VOCs) in a product to contribute to ozone formation, a key factor in air quality management. As environmental concerns grow, regulatory agencies such as the California Air Resources Board (CARB) have tightened limits on VOCs in consumer products, including dry shampoos. For example, California's new VOC limits mandate a maximum of 55% VOCs in dry shampoos, effective from 2023, with even stricter limits expected by 2029.

### SUMMARY

Dry shampoo compositions are provided that may be used for topical application to hair (e.g., human hair) when the hair is in need to cleaning without the need for water. The dry shampoo compositions provided herein may be formulated as a spray or powder. The spray may be under pressure so as to propel the dry shampoo composition onto the hair upon engagement. The dry shampoo compositions provided herein may function, in part, by absorbing excess oil and sebum from the scalp and hair, thereby reducing the appearance of greasiness and extending the time between conventional washes.

The dry shampoo compositions provided herein deliver considerable convenience and versatility, particularly for individuals with limited access to water or those requiring rapid grooming solutions. The dry shampoo compositions provided herein may be subject to repeated use but avoid buildup on the scalp or hair shaft thereby avoiding irritation or obstruction of hair follicles. The dry shampoo compositions provided herein avoid noticeable residue, especially on darker hair. Finally, the dry shampoo compositions provided herein meet or exceed regulatory and safety requirements relevant to dry shampoos.

According to one aspect, the present disclosure provides various dry shampoo compositions that may optionally include one or more of each of the following components: propellants, hair fixatives, emollients, oil-absorbing agents, solvents, preservatives, neutralizing agents, chelating agents, and fragrances.

According to another aspect, the present disclosure provides methods of refreshing or cleansing hair without water. The methods include the steps of providing a dry shampoo composition as described herein and dispensing the dry shampoo composition to the scalp or hair of a subject in need thereof to reduce oiliness and refresh the hair without rinsing.

In yet another aspect, the present disclosure provides a dry shampoo product comprising a container, a dry shampoo composition according to the present disclosure located in an interior product storage region of the container, and a spraying device comprising a stem orifice, an actuator orifice, and optionally a vapor tap orifice.

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

Unless otherwise defined herein, scientific and technical terms used in this application shall have the meanings that are commonly understood by those of ordinary skill in the art. Generally, nomenclature used in connection with, and techniques of chemistry described herein are those well-known and commonly used in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

As used herein, the term “halo” or “halogen” means fluoro, chloro, bromo, or iodo, each of which may be used interchangeably with the terms F, Cl, Br or I, respectively.

Chlorofluorocarbons (CFCs) and hydrochlorofluorocarbons (HCFCs) refer to fully or partly halogenated hydrocarbons that contain carbon (C), hydrogen (H), chlorine (CI), and fluorine (F), produced as volatile derivatives of methane, ethane, and propane. Non-limiting examples of CFCs and HCFCs include trichlorofluoromethane (CFC-11), dichlorodifluoromethane (CFC-12), 1,1,2-trichloro-1,2,2-trifluoroethane (CFC-113), 1,2-dichloro-1,1,2,2-tetrafluoroethane (CFC-114), chloropentafluoroethane (CFC-115), chlorodifluoromethane (HCFC-22), 1,1,1-trifluoro-2,2-dichloroethane (HCFC-123), 1,1-dichloro-1-fluoroethane (HCFC-141b), 1-chloro-1,1-difluoroethane (HCFC-142b), and 2-chloro-1,1,1,2-tetrafluoroethane (HCFC-124),

Hydrofluorocarbons (HFCs) refer to synthetic organic compounds that contain fluorine and hydrogen atoms, and are the most common type of organofluorine compounds. Non-limiting examples of HFCs include trifluoromethane (HFC-23), 1,1,2,2-tetrafluoroethane (HFC-134), 1,1,1,2-tetrafluoroethane (HFC-134a), pentafluoroethane (HFC-125), 1,1,1-trifluoroethane (HFC-143a), 1,1-difluoroethane (HFC-152a), ethoxy-nonafluorobutane (HFE 7200), trans-1,3,3,3-tetrafluoropropene (HFO-1234ze), and perfluorocarbons.

“Global Warming Potential (GWP)” means the radiative forcing impact of one mass-based unit of a given greenhouse gas relative to an equivalent unit of carbon dioxide over a given period of time (e.g., one hundred years). For example, HFC-152a has a GWP of 124, meaning it is 124 times more potent than carbon dioxide over a 100-year period.

The terms “hair-fixative polymer” or “hair fixative” are given their ordinary meaning in the art and generally refer to a film-forming polymer which is soluble in selected solvent(s) (e.g., present in the dry shampoo composition) and is capable of forming a film and/or holding the hair of a user in place after evaporation of the selected solvent(s). Non-limiting examples of hair fixative polymers include octylacrylamide/acrylates/butylaminoethyl/methacrylate copolymer, PVP, VP/VA copolymer, PVP/VA copolymer, acrylates copolymer, polyurethane-14/AMP-acrylates copolymer, esters of PVM/MA copolymer, and combinations thereof. Other suitable hair fixatives will be known to those of ordinary skill in the art.

“Hair Styling Product” means a consumer product that is designed or labeled for application to wet, damp or dry hair to aid in defining, shaping, lifting, styling and/or sculpting of the hair. “Hair Styling Product” includes, but is not limited to, hair balm, clay, cream, creme, curl straightener, gel, liquid, lotion, paste, pomade, putty, root lifter, serum, spray gel, stick, temporary hair straightener, wax, spray products that aid in styling but do not provide finishing of a hair style, and leave-in volumizers, detanglers and/or conditioners that make styling claims.

“Hair Styling Product” does not include “Dry Shampoo,” “Thermal Protectant,” “Hair Mousse,” “Hair Shine,” “Hair Finishing Spray,” or shampoos or conditioners that are rinsed from the hair prior to styling.

“Head space” refers to the safety margin to allow for expansion in a pressurized container.

“Finish” or “finishing” means the maintaining and/or holding of a hairstyle once all styling is complete.

“Styling” means forming, sculpting, or manipulating the hair to alter the hair's shape.

“Maximum Incremental Reactivity (MIR)” refers to the quantifiable metric used to evaluate the ozone-forming potential of a specific volatile organic compound (VOC) when introduced into a controlled mixture of reactive organic gases (e.g., a base ROG mixture). The MIR value is defined as the maximum increase in the mass of ozone produced, expressed in grams of ozone (g O₃), per gram of the VOC added (g VOC). The MIR metric is utilized primarily to assess and compare the relative contribution of different VOCs to photochemical smog formation. The calculation of MIR involves the addition of a defined quantity of the VOC to a Base ROG Mixture, under conditions that replicate atmospheric photochemical reactions, including the presence of sunlight and nitrogen oxides (NOx). The resulting change in ozone concentration, observed within a specified period, is then divided by the mass of the VOC added to determine the MIR value pursuant to the following formula:

MIR=Total Incremental O₃ Formed (g)/VOC Added (g)

MIR values may be determined based on established scientific models, such as those developed or published by the California Air Resources Board (CARB), or other recognized regulatory or scientific authorities. For purposes of the present disclosure, MIR values are used to assess, control, or limit the aggregate ozone-forming potential of compositions containing one or more VOCs.

“Propellant” means a liquefied or compressed gas that is used in whole or in part, such as a cosolvent, to expel a liquid or any other material from the same self-pressurized container or from a separate container.

“Type A Propellant” means a compressed gas such as CO2, N2, N2O, or compressed air, which is used as a propellant, and is either incorporated with the product or contained in a separate chamber within the product's packaging.

“Type B Propellant” means any halocarbon which is used as a propellant including chlorofluorocarbons (CFCs), hydrochlorofluorocarbons (HCFCs), and hydrofluorocarbons (HFCs).

“Type C Propellant” means any propellant which is not a Type A or Type B propellant, including propane, isobutane, n-butane, and dimethyl ether (also known as dimethyl oxide).

“Volatile Organic Compound (VOC)” means any compound containing at least one atom of carbon, excluding carbon monoxide, carbon dioxide, carbonic acid, metallic carbides or carbonates, and ammonium carbonate, and excluding the following:

“VOC Content” means the total weight of VOC in a product expressed as a percentage of the product weight (exclusive of the container or packaging).

The phrases “wt %,” “percent by weight,” “by weight percent” are used interchangeably and refer to the percent of a component based on the total weight of the composition.

### Overview

The present disclosure provides various dry shampoo compositions suitable for a variety of hair care and styling applications. According to one embodiment, the dry shampoo compositions may provide multiple cosmetic and functional benefits to the hair following application. The compositions may be applied to help refresh the appearance and feel of hair by absorbing excess oil and sebum at the scalp and along the hair shaft, thereby extending the time between traditional washes. In addition to oil control, the dry shampoo compositions may improve the visual appearance of the hair by enhancing volume, texture, and body while reducing the appearance of greasiness or flatness. The compositions may include conditioning agents, anti-static compounds, or protective additives such as UV filters to support the overall health and manageability of the hair. Unlike traditional dry shampoos that may leave behind a heavy or chalky residue, the dry shampoo compositions provided herein are formulated to leave a clean, lightweight finish without visible buildup or flaking. The compositions exhibit rapid drying following application and leave the hair soft to the touch with a refreshed, non-tacky feel.

The dry shampoo compositions provided herein exhibit a maximum incremental reactivity (MIR) of 0.75, 0.74, 0.73, 0.72, 0.71, 0.70 or less. According to one embodiment, the dry shampoo compositions may have an MIR of less than about 0.70, less than about 0.65, or less than about 0.60. In some embodiments, the compositions may have an MIR from about 0.40 to about 0.79, about 0.50 to about 0.78, or about 0.50 to about 0.75.

In certain embodiments, the dry shampoo compositions include only components with a Global Warming Potential (GWP) of less than 10. According to one embodiment, the compositions include only components with a GWP of less than about 10, less than about 5, or less than about 2. In certain embodiments, the compositions include only components with a GWP of 0. In certain embodiments, the dry shampoo composition has a GWP equal to or less than 150.

In some embodiments, the dry shampoo compositions are substantially free or completely free of 1,1-difluoroethane (HFC-152a). In some embodiments, the compositions do not include one or more of a Type A, Type B, or Type C propellant. In some embodiments, the compositions may include minimal amounts of one or more of a Type A, Type B, or Type C propellant such that the MIR is maintained at or below 0.75. In some embodiments, the dry shampoo compositions include primarily non-VOC propellants to reduce ozone formation potential while maintaining a MIR of less than 0.75.

Various dry shampoo compositions are provided and may be incorporated in aerosol or non-aerosol delivery formats for use in both consumer and professional hair care products. The dry shampoo compositions of the present disclosure are free or devoid of alcohols. The dry shampoo compositions of the present disclosure may include water-based or water-free formulations optimized for performance, environmental safety, and cosmetic elegance.

### A. Propellant

The dry shampoo compositions provided herein include one or more propellants. Non-limiting examples of propellants include liquefied gas propellants such as alkanes (e.g., ethane, propane, n-butane, isobutane, n-pentane, iso-pentane, blends of hydrocarbons (e.g., C₃-C₇ hydrocarbons), liquefied petroleum gas), and ethers (e.g., dimethyl ether), and compressed gas propellants such as carbon dioxide, nitrous oxide, nitrogen. In some embodiments, the propellant is a liquefied gas propellant. For example, the liquefied gas propellant may be an alkane, an ether, or a combination thereof.

In some embodiments, the propellant includes at least one of an alkane (e.g., a hydrocarbon) or an ether. For example, the propellant may comprise an alkane blend of two or more (i.e., 2, 3, or 4) alkanes. In some embodiments, the propellant includes an alkane blend, the alkane blend comprising a first alkane and a second alkane that is different than the first alkane. For example, the propellant may include a first alkane (e.g., propane) and a second alkane (e.g., butane, such as n-butane, iso-butane). In some embodiments, the propellant includes propane and butane. In some embodiments, the propellant includes a hydrocarbon blend of alkanes (e.g., a mixture of C3-C7 hydrocarbons). In some embodiments, the propellant includes an ether (e.g., dimethyl ether). In some embodiments, the propellant may comprise A-17 (n-butane) available from Aeropres Corp., A-31 (iso-butane) available from Aeropres Corp., A-108 (propane) available from Aeropres Corp., AB-70 (50% propane/50% n-butane alkane blend) available from Aeropres Corp., or dimethyl ether available from Diversified CPC International, Inc.

In some embodiments, the dry shampoo compositions include various weights of propellant as set forth in the ranges in the exemplary embodiments set forth herein. In some embodiments, the dry shampoo compositions include about 10.0% to about 95.0% by weight of the propellant. For example, the dry shampoo compositions may include greater than about 30.0%, about 45.0%, about 40.0%, about 50.0%, about 55.0%, about 60.0%, about 65.0%, about 70.0%, about 75.0%, about 80.0%, or about 85.0% by weight of at least one propellant. In other embodiments, the dry shampoo compositions may include less than about 95.0%, about 90.0%, about 85.0%, about 80.0%, about 75.0%, about 70.0%, about 65.0%, about 60.0%, about 55.0%, about 50.0%, about 45.0%, about 40.0%, about 35.0%, or about 30.0% by weight of at least one propellant. In some embodiments, the dry shampoo compositions may include from about 30.0% to about 95.0%, about 35.0% to about 95.0%, about 35.0% to about 95.0%, about 40.0% to about 95.0%, or about 45.0% to about 95.0% by weight of at least one propellant.

In some embodiments, the dry shampoo compositions include about 10.0% to about 95.0% by weight of the propellant. In some water-based embodiments, the dry shampoo compositions include about 40.0%, about 45.0%, about 50.0%, about 55.0%, about 60.0%, about 65.0%, about 70.0%, about 75.0%, about 80.0%, about 85.0%, about 90.0%, or about 95.0% by weight of at least one propellant. In some embodiments, the dry shampoo compositions may include from about 50.0% to about 95.0% by weight of at least one propellant. In some embodiments, the dry shampoo compositions may include from about 55.0% to about 65.0% by weight of at least one propellant.

### B. Solvents

In some embodiments, the dry shampoo composition includes at least one of a solvent. For example, the dry shampoo composition may comprise a solvent mixture of two or more (i.e., 2, 3, or 4) solvents. In some embodiments, the dry shampoo composition includes a solvent mixture, the solvent mixture comprising a first solvent (e.g., an alcohol) and a second solvent (e.g., water) that is different than the first solvent.

In some embodiments, the dry shampoo composition includes about 1.0% to about 40.0% by weight of the solvent. For example, the dry shampoo composition may comprise less than about 40.0%, less than about 35.0%, less than about 30.0%, less than about 25.0%, less than about 20.0%, or less than about 15.0%. In some embodiments, the dry shampoo may include from about 5.0% to about 40.0%, from about 5.0% to about 35.0%, from about 5.0% to about 30.0%, from about 5.0% to about 25.0%, from about 5.0% to about 20.0%, or about 5.0% to about 15.0% by weight of solvent. In some embodiments, the dry shampoo composition includes about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 21.0%, about 22.0%, about 23.0%, about 24.0%, about 25.0%, about 26.0%, about 27.0%, about 28.0%, about 29.0%, about 30.0%, about 31.0%, about 32.0%, about 33.0%, about 34.0%, about 35.0% by weight of solvent.

In some embodiments, the solvent is an alcohol. Non-limiting examples of alcohols include ethanol, propanol, and butanol. In certain embodiments, the alcohol is propanol (e.g., isopropanol). In some embodiments, the alcohol may comprise 99% isopropanol (isopropyl alcohol—IPA) available from Sigma-Aldrich. In some embodiments, the solvent is an alkane (e.g., isohexadecane, and n-dodecane).

In some embodiments, the dry shampoo composition includes about 5% to about 20% by weight of the alcohol. For example, the dry shampoo composition may comprise less than about 20%, less than about 15%, less than about 12.5%, about 5% to about 15% or about 7.5% to about 12.5% by weight of alcohol. In some embodiments, the dry shampoo composition includes about 5%, about 6%, about 7%, about 8%, about 9%, about 10%, about 11%, about 12%, about 13%, about 14%, or about 15% by weight of alcohol.

In some embodiments, the dry shampoo composition includes about 15% to about 35% by weight of water. For example, the dry shampoo composition may include less than about 30%, less than about 25%, or about 20% to about 25% by weight of water. In some embodiments, the dry shampoo composition includes about 15%, about 16%, about 17%, about 18%, about 19%, about 20%, about 21%, about 22%, about 23%, about 24%, about 25%, about 26%, about 27%, about 28%, about 29%, about 30% or more by weight of water.

### C. Oil-Absorbing Components

In some embodiments, the dry shampoo compositions may include at least one oil-absorbing component. In some embodiments, the oil-absorbing component may be a powder. In certain embodiments, the oil-absorbing component includes a starch (e.g., potato starch, rice starch, tapioca starch, wheat starch, or cornstarch). The oil-absorbing component may include at least one type of starch. For example, it may include a mixture of two or more starches (i.e., two, three, or four starches). In some embodiments, the oil-absorbing component includes a starch mixture comprising a first starch and a second starch that is different from the first starch. For instance, the oil-absorbing component may include a first starch (e.g., rice starch) and a second starch (e.g., potato starch). In other embodiments, the oil-absorbing component includes charcoal, alumina, cellulose (e.g., hydroxyethylcellulose), carbonate, hectorite, clay, silica, capsoil hydrolyzed corn starch, or a combination thereof.

Non-limiting examples of starches include cornstarch, potato starch, tapioca starch, rice starch, wheat starch, and cassava starch. Starches may be characterized by two distinct granule types: A-type starch (AS), with granules larger than 10.0 μm in diameter, and B-type starch (BS), with granules measuring no more than 10.0 μm in diameter. In some embodiments, the starch may also function as an oil absorber. The starch described herein may be modified or unmodified. A modified starch is one that has been derivatized or physically or chemically altered by processes familiar to those skilled in the art, such as esterification, etherification, oxidation, acid hydrolysis, crosslinking, or enzyme conversion. Non-limiting examples of modified starches include aluminum starch octenylsuccinate, aluminum starch octenylsuccinate with acrylates copolymer and magnesium carbonate, sodium starch octenylsuccinate, calcium starch octenylsuccinate (e.g., modified cornstarch), distarch phosphate, hydroxyethyl starch phosphate, hydroxypropyl starch phosphate, sodium carboxymethyl starch, dimethylimidazolidinone starch (e.g., modified rice starch), starch with cetrimonium chloride (e.g., modified rice starch), and sodium starch glycolate. It should be understood, however, that in some embodiments, starch may be replaced by, or used in conjunction with, another oil-absorbing component such as cellulose, maltodextrin, magnesium carbonate, chalk (e.g., calcium carbonate), talc, or fuller's earth. In some embodiments, the component may include DSA 79092 (Oryza sativa (rice) starch, cetrimonium chloride) available from Agrana USA, rice starch NS (dimethylimidazolidinone rice starch) available from Agrana USA, potato starch B available from Roquette, NATRASORB® HFB (aluminum starch octenylsuccinate, acrylates copolymer, magnesium carbonate) available from Nouryon, or FARMAL™ AF 1100 (calcium starch octenylsuccinate), available from Ingredion.

In some embodiments, the oil-absorbing component includes silica (e.g., silica powder). In certain embodiments, the silica includes a first silica and a second silica that is different from the first. Silica (e.g., as a suspending agent, cleaning agent, or oil-absorbing agent) may include Aerosil 200 F available from Evonik, Cabo-O-Sil available from Cabot, Spherilex 10PC available from Evonik, SPHERON P-1500 available from Presperse, LOR Bentone 38V CG (modified hectorite clay including magnesium silicate with lithium and sodium ions). or Solesphere H-53 available from Resifa.

In some embodiments, the dry shampoo composition includes about 0.01% to about 8.0% by weight of silica. For example, the composition may include greater than about 0.01%, greater than about 0.05%, or greater than about 0.1% and less than about 8.0%, less than about 5.0%, about 0.01% to about 8.0%, about 0.01% to about 5.0%, about 0.01% to about 3.0%, about 0.01% to about 2.0%, about 0.01% to about 1.0%, about 0.05% to about 5.0%, about 0.05% to about 4.0%, about 0.05% to about 3.0%, about 0.05% to about 2.0%, about 0.05% to about 1.0%, about 0.1% to about 8.0%, about 0.1% to about 5.0%, about 0.1% to about 3.0%, about 0.1% to about 2.0%, about 0.1% to about 1.0%, about 0.5% to about 5.0%, about 0.5% to about 4.0%, about 0.5% to about 3.0%, about 0.5% to about 2.0%, or about 0.5% to about 1.0% by weight of silica.

In some embodiments, the dry shampoo composition includes about 1.0% to about 20.0% by weight of the oil-absorbing component. For example, the dry shampoo composition may include less than about 20.0%, less than about 15.0%, less than about 12.5%, less than about 10.0%, less than about 9.0%, less than about 8.0%, less than about 7.0%, or less than about 6.0% by weight of oil-absorbing component. In some embodiments, the dry shampoo composition includes about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, or about 15.0% by weight of oil-absorbing component. In some embodiments, the dry shampoo composition includes from about 2.0% to about 6.0% by weight of oil-absorbing component.

In some embodiments, an oil-absorbing component (e.g., a starch and/or silica) and optionally an additive may be combined to form a concentrate. In other embodiments, the concentrate includes an oil-absorbing component (e.g., a starch and/or silica), and optionally silk, zeolite, or a combination thereof. The concentrate may include about 90.0% to about 100.0% by weight of an oil-absorbing component. Alternatively, the concentrate may include about 60.0% to about 100.0%, about 70.0% to about 100.0%, or about 80.0% to about 100.0% by weight of oil-absorbing component. For example, the concentrate may include about 90.0% to about 100.0% by weight of an oil-absorbing component (e.g., approximately 95.0% by weight) and up to about 10.0% by weight of an additive (e.g., approximately 5.0% by weight).

### D. Preservatives

In some embodiments, the dry shampoo compositions may include at least one preservative (e.g., one, two, three, four, five, or more). Suitable preservatives include, but are not limited to, phenoxyethanol, ethylhexylglycerin, methylparaben, propylparaben, DMDM hydantoin, imidazolidinyl urea, diazolidinyl urea, chlorphenesin, caprylyl glycol, benzyl alcohol, dehydroacetic acid, sodium benzoate, potassium sorbate, sorbic acid, and phenylpropanol. These preservatives may be included individually or in combination in an amount effective to inhibit the growth of microorganisms, such as bacteria, yeasts, and molds, thereby enhancing the microbiological stability and shelf life of the dry shampoo compositions.

In some embodiments, they dry shampoo compositions include at least one preservative as set forth in the ranges in the exemplary embodiments set forth herein. In some embodiments, the dry shampoo compositions include from about 0.01% to about 2.0% by weight of the at least one preservative. For example, the dry shampoo compositions may include less than about 2.0%, about 1.5%, about 1.0%, about 0.75%, about 0.50%, or about 0.1% by weight of the at least one preservative. In some embodiments, the dry shampoo compositions include about 0.01%, about 0.05%, about 0.1%, about 0.3%, about 0.5%, about 0.75%, about 1.0%, about 1.5% or about 2.0% by weight of the at least one preservative. In some embodiments, the dry shampoo compositions include about 0.1% to about 2.0% of the at least one preservative.

### E. Hair Fixatives

In some embodiments, the dry shampoo compositions may include at least one hair fixative (e.g., one, two, three, four, five, or more). The hair fixative may function as a binder or film former in addition to a hair fixative. The hair fixative may include one or more film-forming polymers, copolymers, biopolymers, or resins including, but not limited to, Trimethylsiloxysilicate, PVP Butylated, VP/VA copolymer, Octylacrylamide/Acrylates/Butylaminoethyl Methacrylate Copolymers, Acrylates/Octylacrylamide Copolymer, VA/Crotonated/Vinyl Neodecanoate Copolymer, Polyurethane-14, VP/VA Copolymer, PVP, Polyquaternium-46 Copolymer, Butyl Ester of PVM/MA Copolymer, Acrylates/Hydroxyesters Acrylates Copolymer, Acrylates Copolymer, VP/Vinyl Caprolactam/DMAPA Acrylates Copolymer, Vinyl Caprolactam/VP/Dimethylaminoethylmethacrylate Copolymer, and Methacryloyl Ethyl Betaine/Acrylates Copolymer. These polymers may be used alone or in combination to impart desired properties such as hold, flexibility, humidity resistance, and shine to the finished hair style.

In some embodiments, the dry shampoo compositions may include at least one hair fixative in the ranges in the exemplary embodiments set forth herein. In some embodiments, the dry shampoo compositions include from about 0.001% to about 5.0% by weight of the at least one hair fixative. For example, the dry shampoo compositions may include less than about 5.0%, about 4.0%, about 3.0%, about 2.0%, about 1.0%, about 0.5%, about 0.1%, about 0.01%, or about 0.001% by weight of the at least one hair fixative. In some embodiments, the dry shampoo compositions include about from about 0.1% to about 2.0% by weight of the at least one hair fixative.

### F. Neutralizing Agents

In some embodiments, the dry shampoo compositions may include at least one neutralizing agent. When present, the neutralizing agent may be utilized to adjust or stabilize the pH of the dry shampoo compositions, typically to a level that is compatible with both the hair and scalp (usually in the range of pH about 4.5 to about 9.5 or about 5.5 to about 9.5). The at least one neutralizing agent may react with acidic groups (such as in hair fixative components) to enhance solubility, viscosity control, and film formation, thereby improving the spray's performance, such as hold strength, dry time, and resistance to humidity. Common neutralizing agents include, but are not limited to, amines, aminomethyl propanol, potassium hydroxide, sodium hydroxide, triethanolamine, citric acid, lactic acid (88.0% lactic acid; 12.0% water; food chemical codex (FCC)), and any combination thereof selected based on the target pH, compatibility with other ingredients, and desired performance attributes. In some embodiments, the dry shampoo compositions may include citric acid granular anhydrous (fine) which may additionally function as a chelating agent and preservative. By including at least one neutralizing agent, proper neutralization may be achieved to minimize scalp irritation and ensure the stability and clarity of the dry shampoo compositions.

In some embodiments, the dry shampoo compositions may include at least one neutralizing agent in the ranges in the exemplary embodiments set forth herein. In some embodiments, the dry shampoo compositions include from about 0.0001% to about 15.0% by weight of the at least one neutralizing agent. For example, the dry shampoo compositions may include less than about 15.0%, about 12.5%, about 10.0%, about 7.5%, about 5.0%, 2.5%, about 0.50%, about 0.10%, about 0.01%, about 0.001%, or about 0.0001% by weight of the at least one neutralizing agent. In some embodiments, the dry shampoo compositions include about 0.0001% to about 0.1% by weight of the at least one neutralizing agent.

### G. Emollients

In some embodiments, the dry shampoo compositions may include at least one emollient in the ranges in the exemplary embodiments set forth herein. In some embodiments, the at least one emollient may include DL Panthenol (analog of pantothenic acid (vitamin B5) available from Sigma-Aldrich), or Radia 7732 (isopropyl palmitate available from CoastSouthwest) or glycerin (e.g., Refined Glycerine 99.7% MIN USP KSHR available from Wilmar), ZEMEA® (propanediol available from DuPont Tate), C13-C15 alkane (mixture of saturated hydrocarbons), heptyl undecylenate, Ceraphyl 230 (ispropyl isostearate), Cosmosil SBS oil (sea buckthorn seed oil obtained by cold pressing seeds of Hippophaë rhamnoides plant), or any combination thereof.

In some embodiments, the dry shampoo compositions include from about 0.05% to about 6.5% by weight of the at least one emollient. For example, the dry shampoo compositions may include less than about 6.5%, about 6.0%, about 5.0%, about 4.5%, about 4.0%, about 3.5%, about 3.0%, about 2.5%, about 2.0%, about 1.5%, about 1.0%, about 0.5%, or about 0.10%, or about 0.01% by weight of the at least one emollient. In some embodiments, the dry shampoo compositions include about 0.01% to about 6.0% or from about 0.05% to about 5.0% by weight of at least one emollient.

### H. Chelating Agents

In some embodiments, the dry shampoo compositions may include at least one chelating agent. The chelating agent may aid in surfactant performance. At least one suitable chelating agent includes tetrasodium glutamate diacetate or Pollout® Detox available from Chemyunion which includes from about 35.0 wt % to about 63.9 wt % sodium polyitaconate, about 35.0 wt % to about 61.0 wt % sodium citrate and about 0.10 wt % to about 5.0 wt % Sapindus mukorossi fruit extract. In some embodiments, the dry shampoo compositions include from about 0.01% to about 10.0% by weight of at least one chelating agent. In some embodiments, the dry shampoo compositions include up to about 5.0% by weight of the at least one chelating agent. In some embodiments, the dry shampoo compositions include from about 0.05% to about 5.0% by weight of the at least one chelating agent.

### I. Fragrances

In some embodiments, the dry shampoo composition may include one or more fragrances to mask any unpleasant odors and enhance the overall sensory experience for the user. Suitable fragrances include, but are not limited to, terpinolene, α-terpineol, linalool, geraniol, citronellol, limonene, eucalyptol, β-pinene, myrcene, and p-cymene. These fragrance compounds may be selected based on their compatibility with hair care formulations and their ability to impart fresh, herbaceous, citrus, or floral olfactory notes like those of terpinolene. Each of the listed fragrance components may be used individually or in combination to enhance the sensorial profile of the composition, while maintaining stability and performance.

In some embodiments, the dry shampoo compositions may include at least one fragrance in the ranges in the exemplary embodiments set forth herein. In some embodiments, the dry shampoo compositions include from about 0.01% to about 3.0% by weight of the at least one fragrance. For example, the dry shampoo compositions may include less than about 3.0%, about 2.0%, about 1.5%, about 1.0%, about 0.75%, about 0.50%, or about 0.1% by weight of the at least one fragrance. In some embodiments, the dry shampoo composition includes about 0.01%, about 0.05%, about 0.1%, about 0.5%, about 0.75%, about 1.0%, about 1.5% or about 2.0% by weight of the at least one fragrance.

### J. Additives and Other Components

In some embodiments, the dry shampoo compositions may include at least one additive in the ranges in the exemplary embodiments set forth herein.

In some embodiments, the dry shampoo compositions may include at least one texturizing component. The texturizing component may aid in altering the feel, appearance, and structure of the hair. Suitable texturizing components include rice silk, zeolite and silica. When present, the rice silk or other texturizing component may be present in an amount of about 0.01% to about 5.0% by weight.

In some embodiments, the dry shampoo compositions may include at least one emulsifier. In certain embodiments, the hair styling composition may include one or more emulsifiers to facilitate the formation and stabilization of emulsions comprising immiscible phases, such as oil-in-water or water-in-oil systems. Emulsifiers serve to reduce interfacial tension between the aqueous and lipid components, thereby ensuring homogeneous distribution of functional ingredients and enhancing formulation stability over time. Suitable emulsifiers may include PEG-40 hydrogenated castor oil, a nonionic surfactant derived from castor oil that has been ethoxylated and hydrogenated to improve its solubility, mildness, and emulsifying capacity. PEG-40 hydrogenated castor oil functions effectively across a broad pH range and is compatible with a wide variety of oils, fragrances, and conditioning agents. It is particularly useful for solubilizing fragrance oils and lipophilic active ingredients into aqueous systems, providing clarity and stability in transparent formulations. Another suitable emulsifier is C12-C15 Pareth-12, a nonionic ethoxylated alcohol composed of fatty alcohols with 12 to 15 carbon atoms reacted with approximately 12 moles of ethylene oxide. This emulsifier is commonly used in personal care formulations for its ability to provide mild emulsification and a smooth skin or hair feel. In some embodiments, these emulsifiers may be combined to achieve synergistic effects, optimizing emulsion texture, viscosity, and product aesthetics. The inclusion of such emulsifiers may also improve the deposition of actives, enhance rinse-off behavior, and contribute to the overall sensorial profile of the product. The choice and concentration of emulsifier(s) may be selected based on factors such as formulation type (e.g., spray, foam, gel), phase polarity, ingredient solubility, and the desired final appearance and stability of the hair styling product.

In some embodiments, the additive may be a conditioner such as Crotein™ ADW (AMP-isostearoyl hydrolyzed (modified) wheat protein available from Croda Inc.). In some embodiments the additive may impact hair growth such as, for example, Rhodiola extract.

In some embodiments, the dry shampoo compositions may include at least one thickening or rheology agent to enhance the rheological properties, formulation stability, and application aesthetics of the product. While carbomer is a commonly employed synthetic thickener due to its high efficiency and clarity in aqueous systems, alternative thickeners may be utilized to achieve similar or improved performance characteristics, particularly in response to formulation constraints or desired sensory profiles. Suitable alternative synthetic or semi-synthetic thickeners include acrylates/C10-30 alkyl acrylate crosspolymer, which provides excellent viscosity enhancement and emulsion stabilization upon neutralization, as well as polyacrylamide and ammonium acryloyldimethyltaurate/VP copolymer, both of which offer enhanced texture and salt tolerance. In further embodiments, the composition may incorporate naturally derived or bio-based thickeners. Examples include xanthan gum, guar gum, sclerotium gum, gellan gum, and carrageenan, which are polysaccharide thickeners derived from microbial fermentation or botanical sources. The choice of thickening agent may be guided by considerations such as desired formulation transparency, pH compatibility, electrolyte tolerance, processing method, and the target sensory characteristics of the hair styling product.

In some embodiments, the dry shampoo compositions may include a softener, suspending agent, a masking agent (e.g., zeolite), an oil absorber, a friction agent, a UV protection component, or filter (e.g., Benzophenone-4), or damage prevention additive (e.g., sunflower oil).

Other suitable additive components will be known to those of ordinary skill in the art. Non-limiting examples of additive components include: antioxidants; essential oils; waxes; fillers; hair-fixative polymers; deodorizing agents; pediculicides; anti-dandruff agents; cosmetic and/or dermatological active agents including moisturizers, vitamins and their derivatives, essential fatty acids, sunscreens, herb and/or plant extracts; dispersing or suspending agents; anti-static agents; pearlescent aids; opacifiers; coloring agents, pigments, or dyes (e.g., iron oxide); odor neutralizers (e.g., charcoal; ordenone); and sequestering agents; or combinations thereof. In some embodiments, the additive component may be capable of and/or may aid as a suspending agent, a dispersing agent, and/or a cleaning agent.

In some embodiments, the dry shampoo compositions optionally include PVM/MA ((poly-) methyl vinyl ether, maleic anhydride (furan-2,5-dione)) decadiene crosspolymer that may function as a multifunctional ingredient that provides hold, improves texture, and enhances product stability, all while ensuring a smooth and pleasant user experience.

In some embodiments, the dry shampoo compositions optionally includes about 0.01% to about 5.0% by weight of the at least one additive. For example, the dry shampoo compositions may include less than about 5%, less than about 4%, less than about 3%, less than about 2.5%, less than about 2%, less than about 1.5%, less than about 1%, about 0% to about 2.5%, about 0% to about 2%, about 0% to about 1.5%, about 0% to about 1%, about 0.1% to about 2.5%, about 0.1% to about 2%, about 0.1% to about 1.5%, or about 0.1% to about 1% by weight of the at least one additive. In some embodiments, the dry shampoo compositions include about 0.1%, about 0.2%, about 0.3%, about 0.4%, about 0.5%, about 0.6%, about 0.7%, about 0.8%, about 0.9%, or about 1.0% by weight of the at least one additive.

In some embodiments, the dry shampoo compositions does not include a hydrofluorocarbon (HFC), a chlorofluorocarbon (CFC), or a hydrochlorofluorocarbon (HCFC). In certain embodiments, the dry shampoo compositions do not include a fluorocarbon (e.g., a hydrofluorocarbon (HFC)). For example, the dry shampoo compositions may be free of a fluorocarbon (e.g., 1,1 difluoroethane (152a) or 1,1,1,2 tetrafluoroethane (134a).

### Products and Methods

The present disclosure relates to dry shampoo compositions that may be used for topical application to hair (e.g., human hair) when the hair is in need to cleaning without the need for water. According to one embodiment, the dry shampoo compositions provided here may be formulated as a powder. The dry shampoo compositions provided herein may be formulated as a spray. The spray may be under pressure so as to propel the dry shampoo composition onto the hair upon engagement. The dry shampoo compositions provided herein may function, in part, by absorbing excess oil and sebum from the scalp and hair, thereby reducing the appearance of greasiness and extending the time between conventional washes.

The dry shampoo compositions provided herein deliver considerable convenience and versatility, particularly for individuals with limited access to water or those requiring rapid grooming solutions. The dry shampoo compositions provided herein may be subject to repeated use but avoid buildup on the scalp or hair shaft thereby avoiding irritation or obstruction of hair follicles. The dry shampoo compositions provided herein avoid noticeable residue, especially on darker hair. Finally, the dry shampoo compositions provided herein meet or exceed regulatory and safety requirements relevant to dry shampoos.

In some embodiments, the dry shampoo composition is a powder that may be applied using a shaker-type dispenser, such as a container or bottle equipped with apertures at the top (e.g., similar to a salt shaker). In such embodiments, a user may dispense the powder by shaking the container to release a measured amount of the dry shampoo composition directly onto the scalp, particularly targeting the root areas where sebum or oil tends to accumulate. Following application, the powder may be manually massaged into the scalp using the fingers and subsequently distributed throughout the hair using a brush or comb.

In other embodiments, the powdered dry shampoo composition may be applied via a pump or puff dispenser. Such dispensers are configured to emit a fine, controlled burst or cloud of powder when actuated, allowing for targeted application to specific regions of the scalp, including but not limited to the crown, part lines, and frontal hairline (e.g., bangs). The use of a puff dispenser may reduce the likelihood of over-application and minimize product spillage or mess during use.

In further embodiments, the dry shampoo composition may be applied using a brush-on applicator. In such cases, the powdered dry shampoo may be contained within or dispensed through a brush device, such as a cosmetic brush with an integrated powder reservoir. The dry shampoo composition may be released through the bristles and applied directly to the scalp or root area by brushing. This method facilitates precise application, reduces mess, and allows for localized delivery of the product to desired areas of the scalp or hair.

In one embodiment, the dry shampoo composition is suitable to be dispensed via a Bag-on-Valve (BOV) pump system, a type of pressurized dispensing technology commonly employed in aerosol-like containers. The dry shampoo compositions as provided herein may be contained in a sealed bag attached to a valve while propellant is placed outside the sealed bag (i.e., between the bag and the can). The Bag-on-Valve system may utilize compressed gas, such as nitrogen gas, as the propellant, which is non-flammable, inert, and environmentally friendly. The dry shampoo composition may be housed within the sealed, flexible bag which is typically constructed from laminated foil or plastic and positioned inside the can. This configuration allows for 360-degree application and minimizes dry shampoo composition waste by enabling nearly complete evacuation of the contents.

The BOV-pump dry shampoo system may operate by maintaining a pressurized gas environment between the inner pouch and the can wall. When the actuator is engaged, the pressure from the nitrogen compresses the pouch, thereby cleanly dispensing the product without mixing it with the propellant. This separation between the propellant and the formulation helps to preserve the chemical and physical integrity of the dry shampoo composition, making the system particularly advantageous for formulations that are sensitive, highly viscous, or free from preservatives.

In some embodiments, the dry shampoo compositions provided herein may be adapted to and delivered as an aerosol such as an aerosol powder (e.g., include aerosol dry shampoo compositions). In some embodiments, the disclosure relates to a dry shampoo product that includes a container, a dry shampoo composition located in an interior product storage region of the container, and a spraying device. The spraying device may include a stem orifice, an actuator orifice, and optionally a vapor tap orifice. In some embodiments, the dry shampoo product may include a container, a dry shampoo composition located in an interior product storage region of the container, and a spraying device that includes a stem, an actuator, and a housing. According to one embodiment, the dry shampoo composition is stored directly with a gas (e.g., nitrogen gas) in the container. The container may be pressurized with the compressed gas and, as an actuator is pressed, the compressed gas pushes the dry shampoo composition out.

In some embodiments, the housing of the spraying device includes a vapor tap orifice. In certain cases, the vapor tap orifice of the spraying device is about 0.010 to about 0.062 inches in diameter. In some embodiments, the housing of the spraying device includes a stem orifice that is about 0.010 inches to about 0.040 inches in diameter.

In some embodiments, the spraying device is connected to the container by a crimp. In certain cases, the crimp has a depth of about 0.195 inches to about 0.205 inches. In certain cases, the crimp has a depth of up to about 1.10 inch. The crimp may have a diameter of about 1.075 inches to about 1.65 inches. The crimp may have a height of up to about 0.3 inches.

In some embodiments, the container includes a head space of about 15.0% to about 50.0% (e.g., about 20.0% to about 35.0%).

In some embodiments, the container including a dry shampoo composition has a pressure (e.g., at a temperature of about 25° C.) of about 50.0 psi to about 150.0 psi, about 75.0 psi to about 140.0 psi, about 95.0 psi to about 135.0 psi, or about 100.0 psi to about 130.0 psi.

In some embodiments, the spraying device delivers the dry shampoo composition at a rate of at least about 0.25 g/s (e.g., about 0.25 g/s to about 2.00 g/s). The spraying device may deliver the dry shampoo composition at a rate of about 0.25 g/s to about 1.8 g/s, about 0.25 g/s to about 1.6 g/s, about 0.25 g/s to about 1.4 g/s, about 0.25 g/s to about 1.2 g/s, about 0.25 g/s to about 1.0 g/s, about 0.4 g/s to about 1.2 g/s, about 0.5 g/s to about 1.8 g/s, about 0.5 g/s to about 1.6 g/s, about 0.5 g/s to about 1.4 g/s, about 0.5 g/s to about 1.2 g/s, about 0.5 g/s to about 1.0 g/s, about 0.75 g/s to about 1.8 g/s, about 0.75 g/s to about 1.6 g/s, about 0.75 g/s to about 1.4 g/s, about 0.75 g/s to about 1.2 g/s, or about 0.75 g/s to about 1.0 g/s. In some embodiments, the spraying device delivers the dry shampoo composition with a spray diameter of about 1.5 inches to about 3.0 inches at a distance of about 6 inches.

In some embodiments, the spraying device includes an average spray cone angle of from about 25 degrees to about 35 degrees. In some embodiments, the spraying device includes an exit orifice of from about 0.18 mm to about 0.3 mm.

In some embodiments, the dry shampoo compositions provided herein may be delivered via a mechanical sprayer. In such embodiments, the dry shampoo composition is in liquid form an forced through a nozzle and out as a spray or mist when a pump actuator is pressed to build pressure.

It should be understood, however, that a dry shampoo composition may also be provided using alternative delivery methods, such as applying by hand directly to the hair or using a container fitted with a pump to dispense a liquid composition (e.g., atomizer), as will be understood by those of ordinary skill in the art.

Method for cleaning hair are also provided. According to one embodiment, the method of cleaning hair includes the step of introducing the dry shampoo composition to the scalp or hair of a subject in need thereof to reduce oiliness and refresh the hair without rinsing. After introduction, the cleaned hair may be styled. According to one embodiment, the dry shampoo composition is ejected by a product including a container and a spraying device suitable for ejecting the dry shampoo composition. According to one embodiment, the introduction step includes dispensing the product onto the roots and scalp of dry hair, allowing the dry shampoo composition to sit for a brief period to maximize oil absorption, and then distributing the composition by brushing or massaging through the hair. This process not only helps eliminate visible oil but can also impart additional volume and texture to the hair.

### EXEMPLARY EMBODIMENTS

A potential feature of a dry shampoo composition according to exemplary embodiments of the present disclosure is that the composition provides high oil-absorption. A dry shampoo composition, according to exemplary embodiments of the present disclosure, satisfies a long-felt need for a product that includes many, if not all, of the following features: low environmental impact, improved oil-absorption, and improved hair appearance after use.

In some embodiments, the disclosure relates to compositions and products relating to a dry shampoo composition. Such compositions may reduce or eliminate an oily and/or unaesthetic appearance of hair.

In some embodiments, the disclosure relates to a dry shampoo composition including: about 50.0 wt % to about 95.0 wt % of at least one propellant; and about 1.0 wt % to about 10.0 wt % of at least one oil absorbing powder. The dry shampoo may also include one or more additives in an amount of up to about 5.0 wt %.

In some embodiments, the disclosure relates to a dry shampoo composition including: about 75.0 wt % to about 95.0 wt % of at least one propellant; and about 2.5 wt % to about 7.5 wt % of at least one oil absorbing powder. The dry shampoo may also include one or more additives in an amount of from about 1.0 wt % to about 5.0 wt %.

In some embodiments, the disclosure relates to a dry shampoo composition including: about 50 wt % to about 95 wt % dimethyl ether; and an oil-absorbing component comprising about 2.0 wt % to about 6.0 wt % modified rice starch, and about 1.0 wt % to about 8.0 wt % potato starch; about 0.05 wt % to about 0.2 wt % silica; about 1.0 wt % to about 2.0 wt % zeolite; and about 0.1 wt % to about 0.3 wt % panthenol, wherein the composition has a combined maximum incremental reactivity (MIR) of less than about 0.75.

In some embodiments, the disclosure relates to a dry shampoo composition including: about 55.0 wt % to about 65.0 wt % of dimethyl ether; about 25.0 wt % to about 35.0 wt % water; about 0.5 wt % to about 5.0 wt % silica; about 0.30 wt % to about 0.50 wt % phenoxyethanol and ethylhexylglycerin; about 0.5 wt % to about 1.0 wt % terpinolene; about 0.5 wt % to about 2.0 wt % propanediol; about 0.5 wt % to about 2.0 wt % glycerin; and about 0.01 wt % to about 0.05 wt % PVM/MA decadiene crosspolymer, wherein the composition is an aerosol spray having zero global warming potential.

In some embodiments, the disclosure relates to a dry shampoo composition including: about 40.0 wt % to about 90.0 wt % of a first propellant; about 40.0 wt % to about 90.0 wt % of a second propellant; about 1.0 wt % to about 15.0 wt % of a starch (e.g., rice starch); up to about 5.0 wt % silica; and up to about 5.0 wt % panthenol. According to such an embodiment, the MIR is less than 0.69.

In some embodiments, the disclosure relates to a dry shampoo composition including: about 40.0 wt % to about 50.0 wt % butane; about 40.0 wt % to about 50.0 wt % propane; about 4.0 wt % to about 5.0 wt % of a starch (e.g., rice starch); from about 0.2 wt % to about 0.3 wt % silica; and from about 0.2 wt % to about 0.3 wt % panthenol. According to such an embodiment, the MIR is less than 0.69.

In certain embodiments, the dry shampoo composition conforms to the California Code of Regulations, Title 17, Section 94511 (c). For example, the dry shampoo composition (e.g., “Innovative Liquefied Propellant Product”) meets all of the following criteria:
(A) For a product manufactured before Jan. 1, 2029, the Innovative Liquefied Propellant Product achieves a 50 percent or greater reduction in GHG emissions compared to the Representative HFC-152a Product;
(B) For a product manufactured on or after Jan. 1, 2029, the Innovative Liquefied Propellant Product includes only ingredients with a GWP less than 10, or achieves a 90 percent or greater reduction in GHG emissions compared to the Representative HFC-152a Product; and
(C) The ozone-forming potential of the Innovative Liquefied Propellant Product does not exceed that of the Representative HFC-152a Product.

In certain embodiments, the dry shampoo composition has a maximum incremental reactivity (MIR) of equal to or less than the MIR of a representative HFC-152a product formulation of a dry shampoo (55% VOC) provided by the California Air Resources Board (CARB).

In certain embodiments, the dry shampoo composition has a maximum incremental reactivity (MIR) of equal to or less than about 0.7540. For example, the dry shampoo composition may have an MIR of less than about 0.80, less than about 0.78, less than about 0.76, about 0.50 to about 0.80, about 0.50 to about 0.78, or about 0.50 to about 0.76.

In certain embodiments, the dry shampoo composition includes only components with a Global Warming Potential (GWP) of less than 10. For example, the dry shampoo composition includes only components with a Global Warming Potential (GWP) of less than about 10, less than about 5, or less than about 2. In certain embodiments, the dry shampoo composition includes only components with a Global Warming Potential (GWP) of 0.

In certain embodiments, the dry shampoo composition has a Global Warming Potential (GWP) of equal to or less than 150.

In certain embodiments, the dry shampoo composition has a 50% or greater (e.g., a 90% or greater) reduction in greenhouse gas (GHG) emissions compared to a representative HFC-152a product formulation of a dry shampoo (55% VOC) provided by the California Air Resources Board (CARB).

In some embodiments, the dry shampoo composition does not include water. For example, the dry shampoo composition may be substantially free of water. In some embodiments, the dry shampoo composition does not include an alcohol.

In some embodiments, the dry shampoo composition does not include a hydrofluorocarbon (HFC), a chlorofluorocarbon (CFC), or a hydrochlorofluorocarbon (HCFC). In certain embodiments, the dry shampoo composition does not comprise a fluorocarbon (e.g., a hydrofluorocarbon (HFC)). For example, the dry shampoo composition may be substantially free of a fluorocarbon (e.g., 1,1 difluoroethane (152a) or 1,1,1,2 tetrafluoroethane (134a).

### EXAMPLES

The examples and preparations provided below further illustrate and exemplify particular aspects of embodiments of the disclosure. It is to be understood that the scope of the present disclosure is not limited in any way by the scope of the following examples.

### Example 1

An exemplary dry shampoo in accordance with certain aspects of the present disclosure is provided in Table 1. The MIR was 0.75. Each component had a GWP of 0. Thus, the dry shampoo composition provided a GHG emission of 0.

TABLE 1
Dry Shampoo Composition

| Name | Function | wt % |
|---|---|---|
| DIMETHYL ETHER (DME) | Propellant | 92.50 |
| MODIFIED RICE STARCH | Oil-Absorbing Component | 5.60 |
| SILICA (AEROSIL 200 F) | Additive | 0.10 |
| ZEOLITE | Additive | 1.63 |
| PANTHENOL | Additive | 0.17 |

### Example 2

An exemplary formulation in accordance with certain aspects of the present disclosure is provided in Table 2. The MIR was 0.75. Each component had a GWP of 0. Thus, the dry shampoo composition provided a GHG emission of 0.

TABLE 2
Dry Shampoo Composition

| Name | Function | wt % |
|---|---|---|
| DIMETHYL ETHER (DME) | Propellant | 92.50 |
| POTATO STARCH | Oil-Absorbing Component | 5.60 |
| SILICA (AEROSIL 200 F) | Additive | 0.10 |
| ZEOLITE | Additive | 1.63 |
| PANTHENOL | Additive | 0.17 |

### Example 3

An exemplary formulation in accordance with certain aspects of the present disclosure is provided in Table 3. The MIR was 0.73. Each component had a GWP of 0. Thus, the dry shampoo composition provided a GHG emission of 0.

TABLE 3
Dry Shampoo Composition

| Name | Function | wt % |
|---|---|---|
| DIMETHYL ETHER (DME) | Propellant | 92.50 |
| POTATO STARCH | Oil-Absorbing Component | 1.87 |
| SILICA (AEROSIL 200 F) | Additive | 0.10 |
| ZEOLITE | Additive | 1.63 |
| PANTHENOL | Additive | 0.17 |
| MODIFIED RICE STARCH | Oil-Absorbing Component | 3.74 |

### Example 4

An exemplary formulation in accordance with certain aspects of the present disclosure is provided in Table 4. The MIR was 0.75. Each component had a GWP of 0. Thus, the dry shampoo composition provided a GHG emission of 0.

TABLE 4
Dry Shampoo Composition

| Name | Function | wt % |
|---|---|---|
| DIMETHYL ETHER (DME) | Propellant | 92.50 |
| POTATO STARCH | Oil-Absorbing Component | 1.87 |
| SILICA (AEROSIL 200 F) | Additive | 0.10 |
| SILICA (SPHERON P-1500) | Additive | 1.63 |
| PANTHENOL | Additive | 0.17 |
| MODIFIED RICE STARCH | Oil-Absorbing Component | 2.00 |
| RICE SILK | Additive | 1.74 |

### Example 5

An exemplary formulation in accordance with certain aspects of the present disclosure is provided in Table 5. The MIR was 0.57. Each component had a GWP of 0. Thus, the dry shampoo composition provided a GHG emission of 0.

TABLE 5
Dry Shampoo Composition

| Name | Function | wt % |
|---|---|---|
| DIMETHYL ETHER (DME) | Propellant | 60.00 |
| WATER | Carrier | 32.02 |
| PHENOXYETHANOL & | Additive | 0.40 |
| ETHYLHEXYLGLYCERIN | | |
| PVM/MA DECADIENE CROSSPOLYMER | Additive | 0.02 |
| SODIUM HYDROXIDE | Additive | 0.01 |
| GLYCERIN | Additive | 0.20 |
| PROPANEDIOL | Additive | 1.00 |
| TETRASODIUM GLUTAMATE ACETATE | Additive | 0.10 |
| C13-15 ALKANE (AND) HEPTYL | Additive | 1.00 |
| UNDECYLENATE | | |
| TERPINOLENE | Additive | 0.75 |
| SILICA (SPHERON P-1500) | Oil-Absorbing | 4.00 |
| | Component | |
| SILICA (SOLESPHERE H-53) | Oil-Absorbing | 0.50 |
| | Component | |

### Comparative Example 1

A comparative dry shampoo composition is provided in Table 6 (referred to as Comparative Dry Shampoo Composition #1). The formulation is provided in Table 6. The MIR of Comparative Dry Shampoo Composition #1 was 0.72. Each component had a GWP of 0. Thus, the dry shampoo composition provided a GHG emission of 0.

TABLE 6
Comparative Dry Shampoo Composition #1

| Name | Function | wt % |
|---|---|---|
| ALKANE BLEND (AB70) | Propellant | 76.79666 |
| ISOPROPYL ALCOHOL | Solvent | 2.79 |
| ETHANOL | Solvent | 0.0015 |
| DI WATER | Solvent | 10.00 |
| TAPIOCA STARCH | Oil-Absorbing Component | 10.00 |
| PROPANEDIOL | Additive | 0.12 |
| GLYCERIN | Additive | 0.058 |
| PANTHENOL | Additive | 0.023 |
| MODIFIED WHEAT PROTEIN | Additive | 0.00084 |
| FRAGRANCE | Additive | 0.21 |

Comparative Dry Shampoo Composition #1 exhibited poor performance with respect to long drying time, minimal oil-absorbing, and high humidity. The poor performance of Comparative Dry Shampoo Composition #1 is believed to be the result of proportionally high levels of water (i.e., moisture excess).

### Comparative Example 2

A comparative formulation is provided in Table 7 (referred to as Comparative Dry Shampoo Composition #2). The MIR of Comparative Dry Shampoo Composition #2 was 0.79. Comparative Dry Shampoo Composition #2 provided a GWP of 3844 per 100-gram product.

TABLE 7
Comparative Dry Shampoo Composition #2

| Name | Function | wt % |
|---|---|---|
| HFC 152a | Propellant | 31.00 |
| ISO-BUTANE | Propellant | 39.00 |
| ETHANOL | Solvent | 15.32 |
| RICE STARCH | Oil-Absorbing | 12.00 |
| | Component | |
| MODIFIED STARCH (NATRASORB HFB) | Oil-Absorbing | 1.50 |
| | Component | |
| PVP/VA COPOLYMER | Additive | 0.15 |
| FRAGRANCE | Additive | 1.00 |
| ETHYLHEXYL METHOXYCINNAMATE | Additive | 0.020 |
| PANTHENOL | Additive | 0.0030 |
| RHODIOLA EXTRACT | Additive | 0.0030 |
| VITAMIN (BV-OSC) | Additive | 0.0030 |

Comparative Dry Shampoo Composition #2 contained a high level of flammable propellants, including isobutane and HFC-152a, which raised safety concerns during use and storage. The high global warming potential and volatile organic compound content made Comparative Dry Shampoo Composition #2 environmentally unfriendly and likely non-compliant with current regulations in regions such as California and the European Union. The ethanol concentration was over 15 percent, which causes excessive dryness and irritation to the scalp and hair. Oil-absorbing powders were present at over 13 percent, increasing the likelihood of visible residue, especially on dark hair.

### Comparative Example 3

A comparative formulation is provided in Table 8 (referred to as Comparative Dry Shampoo Composition #3). The MIR of Comparative Dry Shampoo Composition #3 was 0.73. Comparative Dry Shampoo Composition #3 provided a GWP of 3596 per 100-gram product.

TABLE 8
Comparative Dry Shampoo Composition #3

| Name | Function | wt % |
|---|---|---|
| HFC 152a | Propellant | 29.00 |
| ALKANE BLEND (AB70) | Propellant | 24.00 |
| ETHANOL | Solvent | 30.59 |
| ISOHEXADECANE | Solvent | 4.63 |
| DI WATER | Solvent | 0.10 |
| RICE STARCH | Oil-Absorbing | 10.00 |
| | Component | |
| DECAMETHYLCYCLO- | Additive | 1.00 |
| PENTASILOXANE | | |
| PROPANEDIOL | Additive | 0.21 |
| GLYCERIN | Additive | 0.12 |
| PANTHENOL | Additive | 0.26 |
| MODIFIED WHEAT PROTEIN | Additive | 0.10 |
| FRAGRANCE | Additive | 0.00020 |

Comparative Dry Shampoo Composition #3 provided poor performance with respect to long drying time, and minimal oil-absorbing. The poor performance of Comparative Dry Shampoo Composition #3 is believed to have been the result of high levels of solvent (i.e., moisture excess).

Comparative Dry Shampoo Composition #3 also exhibited several performance and formulation drawbacks. Specifically, Comparative Dry Shampoo Composition #3 contained over 50 percent flammable propellants, including an alkane blend and HFC-152a, which posed fire risks and raised environmental concerns due to high global warming potential and VOC content. The ethanol concentration exceeded 30 percent, contributing to excessive dryness and potential scalp irritation. Although isohexadecane improved spreadability, the combination with ethanol may have led to phase instability and did not compensate for the absence of conditioning agents. Rice starch was the sole absorbent and when combined with the other components potentially leaves visible residue on dark hair in the absence of complementary powders to enhance oil control. Functional additives such as panthenol and proteins were present but were not strongly supported by film-formers or humectants. Additionally, the fragrance level was extremely low and may have been insufficient to deliver a noticeable sensory effect over the propellants utilized. Overall, the formula lacked balance between performance, user comfort, and regulatory compliance.

### STATEMENTS OF THE DISCLOSURE

The following summary presents certain aspects of the present disclosure and is not intended to limit the scope of the invention as defined by the claims.
Statement 1. A dry shampoo composition, the composition including:
about 50.0 wt % to about 95 wt % of a liquefied gas propellant; and
about 5.0 wt % to about 50.0 wt % of a concentrate, the concentrate comprising an oil-absorbing component,
wherein the composition has a combined maximum incremental reactivity (MIR) of less than about 0.76.
Statement 2. The composition of statement 1, wherein the liquefied gas propellant includes an alkane, an ether, or a combination thereof.
Statement 3. The composition of statements 1 or 2, wherein the propellant includes an alkane selected from the group consisting of ethane, propane, butane (e.g., n-butane or iso-butane), pentane (e.g., n-pentane, isopentane), blends of hydrocarbons (e.g., C3-C7 hydrocarbons), and combinations thereof.
Statement 4. The composition of any one of the preceding statements, wherein the liquefied gas propellant includes a first alkane (e.g., propane) and a second alkane (e.g., butane, such as n-butane or iso-butane).
Statement 5. The composition of statements 1 or 2, wherein the liquefied gas propellant includes an ether (e.g., dimethyl ether).
Statement 6. The composition of any one of the preceding statements, wherein the composition includes about 75% to about 95% by weight of the propellant.
Statement 7. The composition of any one of the preceding statements, wherein the oil-absorbing component includes a starch (e.g., potato starch, rice starch, tapioca starch, wheat starch, or corn starch).
Statement 8. The composition any one of statements 1 to 6, wherein the oil-absorbing component includes a starch mixture, the starch mixture comprising a first starch and a second starch that is different than the first starch.
Statement 9. The composition of any one of the preceding statements, wherein the oil-absorbing component includes a silica.
Statement 10. The composition any one of statements 1 to 8, wherein the oil-absorbing component includes a silica mixture, the starch mixture comprising a first silica and a second silica that is different than the first starch.
Statement 11. The composition of any one of the preceding statements, wherein the composition includes about 1 wt % to about 10 wt % of the oil-absorbing component.
Statement 12. The composition of any one of the preceding statements, wherein the composition includes about 5 wt % to about 25 wt % of the concentrate.
Statement 13. The composition of any one of the preceding statements, wherein the composition further includes an additive selected from the group consisting of an emollient, a fragrance, a hair fixative, a plasticizer, a humectant, a conditioner, a softener, suspending agent, a masking agent, an oil absorber, a friction agent, a UV filter, a pH adjuster, and any combination thereof.
Statement 14. The composition of any one of the preceding statements, wherein the composition is substantially free of water or solvent.
Statement 15. The composition of any one of statements 1 to 13, wherein the composition further includes a solvent (e.g., water).
Statement 16. The composition of any one of the preceding statements, wherein the composition is substantially free of an alcohol (e.g., ethanol, propanol, or butanol).
Statement 17. The composition of any one of the preceding statements, wherein the composition is substantially free of a fluorocarbon (e.g., 1,1 difluoroethane (152a) or 1,1,1,2 tetrafluoroethane (134a)).
Statement 18. A dry shampoo composition, the composition comprising:
about 90.0 wt % to about 95.0 wt % propellant, wherein the propellant is dimethyl ether;
about 2.5 wt % to about 7.5 wt % rice starch; and
about 0.01 wt % to about 0.5 wt % silica;
wherein the composition has a combined maximum incremental reactivity (MIR) of about 0.50 to about 0.76.
Statement 19. A dry shampoo composition, the composition comprising:
about 90.0 wt % to about 95.0 wt % propellant, wherein the propellant is dimethyl ether;
about 2.5 wt % to about 7.5 wt % potato starch; and
about 0.01 wt % to about 0.5 wt % silica;
wherein the composition has a combined maximum incremental reactivity (MIR) of about 0.50 to about 0.76.
Statement 20. A dry shampoo composition, the composition comprising:
about 90.0 wt % to about 95.0 wt % of a propellant, wherein the propellant is dimethyl ether;
about 1.0 wt % to about 5.0 wt % rice starch;
about 1.0 wt % to about 8.0 wt % potato starch; and
about 0.01 wt % to about 0.5 wt % silica;
wherein the composition has a combined maximum incremental reactivity (MIR) of about 0.50 to about 0.76.
Statement 21. A dry shampoo composition, the composition comprising:
about 90.0 wt % to about 95 wt % propellant, wherein the propellant is dimethyl ether;
about 1.0 wt % to about 5.0 wt % rice starch;
about 1.0 wt % to about 8.0 wt % potato starch; and
about 0.01 wt % to about 2.5 wt % of a first silica and a second silica that is different from the first silica;
wherein the composition has a combined maximum incremental reactivity (MIR) of about 0.50 to about 0.76.
Statement 22. A dry shampoo composition, the composition comprising:
about 55.0 wt % to about 65.0 wt % of a propellant, wherein the propellant is dimethyl ether;
about 25.0 wt % to about 35.0 wt %; and
about 0.10 wt % to about 7.5 wt % of a first silica and a second silica that is different from the first silica;
wherein the composition has a combined maximum incremental reactivity (MIR) of about 0.50 to about 0.76.
Statement 23. A dry shampoo product comprising:
a container,
the dry shampoo composition according to any one of statements 1-22 located in an interior product storage region of the container, and
a spraying device comprising a stem orifice, an actuator orifice, a body orifice of about 0.010 inches to about 0.062 inches, and optionally a vapor tap orifice.
Statement 24. The dry shampoo product of statement 23, wherein the stem orifice is about 0.010 to about 0.040 inches in diameter.
Statement 25. The dry shampoo product of statement 23 or 24, wherein the actuator orifice is about 0.010 to about 0.030 inches in diameter.
Statement 26. The dry shampoo product of any one of statements 23 to 25, wherein the vapor tap orifice is about 0.010 to about 0.062 inches in diameter.
Statement 27. The dry shampoo product of any one of statements 23 to 26, wherein the spraying device delivers the dry shampoo composition at a rate of about 0.50 g/s to about 2.0 g/s.
Statement 28. A dry shampoo composition comprising by weight percent of the total composition:
about 50% to about 95 wt % dimethyl ether; and
an oil-absorbing component comprising:
   about 2.0 wt % to about 6.0 wt % rice starch, and
   about 1.0 wt % to about 8.0 wt % potato starch;
about 0.05 wt % to about 0.2 wt % silica;
about 1.0 wt % to about 2.0 wt % zeolite; and
about 0.1 wt % to about 0.3 wt % panthenol,
wherein the composition has a combined maximum incremental reactivity (MIR) of less than about 0.75.
Statement 29. The dry shampoo composition of statement 28, wherein the silica is present in an amount of about 0.10 wt %.
Statement 30. The dry shampoo composition of statement 28, wherein the potato starch is present in an amount of about 1.0 wt % to about 6.0 wt %.
Statement 31. The dry shampoo composition of claim 30, wherein the rice starch is modified rice starch comprising cetrimonium chloride.
Statement 32. The dry shampoo composition of statement 28, wherein the weight ratio of rice starch to potato starch is from about 1.5:1 to about 2.5:1.
Statement 33. The dry shampoo composition of statement 28, wherein the dimethyl ether is present in an amount of about 92.5 wt %.
Statement 34. The dry shampoo composition of statement 28, wherein all components have a global warming potential of zero.
Statement 35. The dry shampoo composition of statement 28, wherein the composition is free of alcohol, water, smectite clay, hydrophobic emollients, and talc.
Statement 36. The dry shampoo composition of statement 28, wherein the composition is contained within a compressed-gas aerosol dispenser.
Statement 37. The dry shampoo composition of statement 28, further comprising PVM/MA decadiene crosspolymer in an amount of about 0.01 wt % to about 0.05 wt %.
Statement 38. The dry shampoo composition of statement 28, wherein panthenol is included as a conditioning agent to improve combability and shine.
Statement 39. The dry shampoo composition of statement 28, wherein the zeolite is in amount of about 1.6 wt %.
Statement 40. A dry shampoo composition comprising by weight percent of the total composition:
about 55.0 wt % to about 65.0 wt % of dimethyl ether;
about 25.0 wt % to about 35.0 wt % water;
about 0.50 wt % to about 5.0 wt % silica;
about 0.30 wt % to about 0.50 wt % phenoxyethanol and ethylhexylglycerin;
about 0.50 wt % to about 1.0 wt % terpinolene;
about 0.50 wt % to about 2.0 wt % propanediol;
about 0.50 wt % to about 2.0 wt % glycerin; and
about 0.01 wt % to about 0.05 wt % PVM/MA decadiene crosspolymer,
wherein the composition is an aerosol spray having zero global warming potential.
Statement 41. The dry shampoo composition of statement 40, wherein the silica is present in an amount of from about 3.0 wt % to about 5.0 wt %.
Statement 42. The dry shampoo composition of statement 40, further comprising tetrasodium glutamate diacetate as a chelating agent in an amount of about 0.01 wt % to about 1.0 wt %.
Statement 43. The dry shampoo composition of statement 40, further comprising sodium hydroxide to adjust pH to a range of about 6.0 to about 7.5.
Statement 44. The dry shampoo composition of statement 40, wherein the composition contains no volatile organic compounds (VOCs).
Statement 45. The dry shampoo composition of statement 40, wherein the dry shampoo composition is biodegradable and non-flammable.
Statement 46. A method of refreshing or cleansing hair without water, the method comprising:
providing a dry shampoo composition; and
dispensing the dry shampoo composition to the scalp or hair of a subject in need thereof to reduce oiliness and refresh the hair without rinsing,
wherein the dry shampoo composition comprises:
   about 50% to about 95 wt % dimethyl ether; and
   an oil-absorbing component comprising:
      about 2.0 wt % to about 6.0 wt % rice starch, and
      about 1.0 wt % to about 8.0 wt % potato starch;
   about 0.05 wt % to about 0.2 wt % silica;
   about 1.0 wt % to about 2.0 wt % zeolite; and
   about 0.1 wt % to about 0.3 wt % panthenol,
wherein the composition exhibits a maximum incremental reactivity (MIR) value of less than 0.75; or
wherein the dry shampoo composition comprises:
   about 55.0 wt % to about 65.0 wt % of dimethyl ether;
   about 25.0 wt % to about 35.0 wt % water;
   about 0.50 wt % to about 5.0 wt % silica;
   about 0.30 wt % to about 0.50 wt % phenoxyethanol and ethylhexylglycerin;
   about 0.50 wt % to about 1.0 wt % terpinolene;
   about 0.50 wt % to about 2.0 wt % propanediol;
   about 0.50 wt % to about 2.0 wt % glycerin; and
about 0.01 wt % to about 0.05 wt % PVM/MA decadiene crosspolymer,
wherein the composition is an aerosol spray having zero global warming potential, and
wherein each wt % is based on the total weight of the respective dry shampoo composition.
Statement 47. The method of statement 46, wherein dispensing the dry shampoo composition comprises spraying the composition from a pressurized container through a spraying device, the spraying device comprising a vapor tap orifice having a diameter of about 0.010 inches to about 0.062 inches, and wherein the composition is dispensed at a rate of about 0.50 g/s to about 2.0 g/s.

What is claimed is:

1. A dry shampoo composition comprising by weight percent of the total composition:
about 50 wt % to about 95 wt % dimethyl ether; and
an oil-absorbing component comprising:
about 2.0 wt % to about 6.0 wt % rice starch, and
about 1.0 wt % to about 8.0 wt % potato starch;
about 0.05 wt % to about 0.2 wt % silica;
about 1.0 wt % to about 2.0 wt % zeolite; and
about 0.1 wt % to about 0.3 wt % panthenol,
wherein the composition has a combined maximum incremental reactivity (MIR) of less than about 0.75.

2. The dry shampoo composition of claim 1, wherein the silica is present in an amount of about 0.10 wt %.

3. The dry shampoo composition of claim 1, wherein the potato starch is present in an amount of about 1.0 wt % to about 6.0 wt %.

4. The dry shampoo composition of claim 3, wherein the rice starch is modified rice starch comprising cetrimonium chloride.

5. The dry shampoo composition of claim 1, wherein the weight ratio of rice starch to potato starch is from about 1.5:1 to about 2.5:1.

6. The dry shampoo composition of claim 1, wherein the dimethyl ether is present in an amount of about 92.5 wt %.

7. The dry shampoo composition of claim 1, wherein all components have a global warming potential of zero.

8. The dry shampoo composition of claim 1, wherein the composition is free of alcohol, water, smectite clay, hydrophobic emollients, and talc.

9. The dry shampoo composition of claim 1, wherein the composition is contained within a compressed-gas aerosol dispenser.

10. The dry shampoo composition of claim 1, further comprising PVM/MA decadiene crosspolymer in an amount of about 0.01 wt % to about 0.05 wt %.

11. The dry shampoo composition of claim 1, wherein panthenol is included as a conditioning agent to improve combability and shine.

12. The dry shampoo composition of claim 1, wherein the zeolite is in amount of about 1.6 wt %.

13. A dry shampoo composition comprising by weight percent of the total composition:
about 55.0 wt % to about 65.0 wt % of dimethyl ether;
about 25.0 wt % to about 35.0 wt % water;
about 0.50 wt % to about 5.0 wt % silica;
about 0.30 wt % to about 0.50 wt % phenoxyethanol and ethylhexylglycerin;
about 0.50 wt % to about 1.0 wt % terpinolene;
about 0.50 wt % to about 2.0 wt % propanediol;
about 0.50 wt % to about 2.0 wt % glycerin; and
about 0.01 wt % to about 0.05 wt % PVM/MA decadiene crosspolymer,
wherein the composition is an aerosol spray having zero global warming potential.

14. The dry shampoo composition of claim 13, wherein the silica is present in an amount of from about 3.0 wt % to about 5.0 wt %.

15. The dry shampoo composition of claim 13, further comprising tetrasodium glutamate diacetate as a chelating agent in an amount of about 0.01 wt % to about 1.0 wt %.

16. The dry shampoo composition of claim 13, further comprising sodium hydroxide to adjust pH to a range of about 6.0 to about 7.5.

17. The dry shampoo composition of claim 13, wherein the composition contains no volatile organic compounds (VOCs).

18. The dry shampoo composition of claim 13, wherein the dry shampoo composition is biodegradable and non-flammable.

19. A method of refreshing or cleansing hair without water, the method comprising:
providing a dry shampoo composition; and
dispensing the dry shampoo composition to the scalp or hair of a subject in need thereof to reduce oiliness and refresh the hair without rinsing,
wherein the dry shampoo composition comprises:
about 50 wt % to about 95 wt % dimethyl ether; and
an oil-absorbing component comprising:
about 2.0 wt % to about 6.0 wt % rice starch, and
about 1.0 wt % to about 8.0 wt % potato starch;
about 0.05 wt % to about 0.2 wt % silica;
about 1.0 wt % to about 2.0 wt % zeolite; and
about 0.1 wt % to about 0.3 wt % panthenol,
wherein the composition exhibits a maximum incremental reactivity (MIR) value of less than 0.75; or
wherein the dry shampoo composition comprises:
about 55.0 wt % to about 65.0 wt % of dimethyl ether;
about 25.0 wt % to about 35.0 wt % water;
about 0.50 wt % to about 5.0 wt % silica;
about 0.30 wt % to about 0.50 wt % phenoxyethanol and ethylhexylglycerin;
about 0.50 wt % to about 1.0 wt % terpinolene;
about 0.50 wt % to about 2.0 wt % propanediol;
about 0.50 wt % to about 2.0 wt % glycerin; and
about 0.01 wt % to about 0.05 wt % PVM/MA decadiene crosspolymer,
wherein the composition is an aerosol spray having zero global warming potential, and
wherein each wt % is based on the total weight of the respective dry shampoo composition.

20. The method of claim 19, wherein dispensing the dry shampoo composition comprises spraying the composition from a pressurized container through a spraying device, the spraying device comprising a vapor tap orifice having a diameter of about 0.010 inches to about 0.062 inches, and wherein the composition is dispensed at a rate of about 0.50 g/s to about 2.0 g/s.
